(19) United States
(12) Patent Application Publication
(10) Pub. No.: US 2007/0123551 A1
(43) Pub. Date: May 31, 2007

(54) Use of metabotropic glutamarate receptor 5 (mglur5) antagonists for the treatment of gastrointestinal disorders

(76) Inventors: Hakan Larsson, Molndal (SE); Anders Lehmann, Molndal (SE); Erik Lindstrom, Molndal (SE)

Correspondence Address: WHITE & CASE LLP;PATENT DEPARTMENT, 1155 AVENUE OF THE AMERICAS, NEW YORK, NY, 10036, US

(21) Appl. No.: 10/582,182

(22) PCT Filed: Dec. 7, 2004
(86) PCT No.: PCT/SE04/01819

§ 371 Date: Jun. 8, 2006

(30) Foreign Application Priority Data
Dec. 17, 2003 (SE) 0303419-6

(51) Int. Cl. A61K 31/44

(52) U.S. Cl. 514/277

(57) ABSTRACT

The present invention relates to the use of metabotropic glutamate receptor 5 (mGluR5) antagonists for the treatment of functional gastrointestinal disorders, such as functional dyspepsia.

### FIELD OF THE INVENTION

The present invention relates to the use of metabotropic glutamate receptor 5 (mGluR5) antagonists for the treatment of functional gastrointestinal disorders, such as functional dyspepsia.

### BACKGROUND OF THE INVENTION

Functional gastrointestinal disorders, such as functional dyspepsia, can be defined in accordance with Thompson W G, Longstreth G F, Drossman D A, Heaton K W, Irvine E J, Mueller-Lissner S A. C. Functional Bowel Disorders and Functional Abdominal Pain. In: Drossman D A, Talley N J, Thompson W G, Whitehead W E, Coraziarri E, eds. Rome II: Functional Gastrointestinal Disorders: Diagnosis, Pathophysiology and Treatment. 2 ed. McLean, Va.: Degnon Associates, Inc.; 2000:351-432 and Drossman D A, Corazziari E, Talley N J, Thompson W G and Whitehead W E. Rome II: A multinational consensus document on Functional Gastrointestinal Disorders. Gut 45(Suppl.2), II1-II81.9-1-1999.

The metabotropic glutamate receptors (mGluR) are G-protein coupled receptors that are involved in the regulation and activity of many synapses in the central nervous system (CNS). Eight metabotropic glutamate receptor subtypes have been identified and are subdivided into three groups based on sequence similarity. Group I consists of mGluR1 and mGluR5. These receptors activate phospholipase C and increase neuronal excitability. Group II, consisting of mGluR2 and mGluR3 as well as group III, consisting of mGluR4, mGluR6, mGluR7 and mGluR8 are capable of inhibiting adenylyl cyclase activity and reduce synaptic transmission. Several of the receptors also exist in various isoforms, occurring by alternative splicing (Chen, C-Y et al., Journal of Physiology (2002), 538.3, pp. 773-786; Pin, J-P et al., European Journal of Pharmacology (1999), 375, pp. 277-294; Brdäuner-Osborne, H et al. Journal of Medicinal Chemistry (2000), 43, pp. 2609-2645; Schoepp, D. D, Jane D. E. Monn J. A. Neuropharmacology (1999), 38, pp. 1431-1476).

The object of the present invention was to find a new way for the treatment of functional gastrointestinal disorders, such as functional dyspepsia.

### OUTLINE OF THE INVENTION

Metabotropic glutamate receptor 5 (mGluR5) antagonists are useful for the treatment of functional gastrointestinal disorders, such as functional dyspepsia.

Consequently, the present invention is directed to the use of a metabotropic glutamate receptor 5 antagonist for the manufacture of a medicament for the treatment of functional gastrointestinal disorders, such as functional dyspepsia.

Functional dyspepsia refers to pain or discomfort centered in the upper abdomen. Discomfort may be characterized by or combined with upper abdominal fullness, early satiety, bloating or nausea. Etiologically, patients with functional dyspepsia can be divided into two groups:
1—Those with an identifiable pathophysiological or microbiologic abnormality of uncertain clinical relevance (e.g. Helicobacter pylori gastritis, histological duodenitis, gallstones, visceral hypersensitivity, gastroduodenal dysmotility)
2—Patients with no identifiable explanation for the symptoms.

Functional dyspepsia can be diagnosed according to the following:

At least 12 weeks, which need not be consecutive within the preceding 12 months of
1—Persistent or recurrent dyspepsia (pain or discomfort centered in the upper abdomen) and
2—No evidence of organic disease (including at upper endoscopy) that is likely to explain the symptoms and
3—No evidence that dyspepsia is exclusively relieved by defecation or associated with the onset of a change in stool frequency or form.

Functional dyspepsia can be divided into subsets based on distinctive symptom patterns, such as ulcer-like dyspepsia, dysmotility-like dyspepsia and unspecified (non-specific) dyspepsia.

Currently existing therapy of functional dyspepsia is largely empirical and directed towards relief of prominent symptoms. The most commonly used therapies still include antidepressants.

For the purpose of this invention, the term “antagonist” should be understood as including full antagonists, inverse agonists, non-competitive antagonists or competitive antagonists, as well as partial antagonists, whereby a “partial antagonist” should be understood as a compound capable of partially, but not fully, in-activating the metabotropic glutamate receptor 5.

The present invention is directed to the use of any mGluR5 antagonist which has a therapeutic effect in functional gastrointestinal disorders, such as functional dyspepsia.

The term “therapy” and/or “treatment” also includes “prophylaxis” unless there are specific indications to the contrary. The terms “therapeutic” and “therapeutically” should be construed accordingly.

The term “therapeutic effect” is defined herein as an effect favourable in the context of the therapy and/or treatment of functional gastrointestinal disorders, such as functional dyspepsia.

One example of a compound having antagonistic affinity to metabotropic glutamate receptor 5, thereby being useful in accordance with the invention, is the compound 2-methyl-6-(phenylethynyl)-pyridine (often abbreviated MPEP). MPEP is commercially available from e.g. Tocris, or may be synthesized according to well-known procedures such as disclosed by K. Sonogashira et al. in Tetrahedron Lett. (1975), 50, 4467-4470.

Pharmaceutical Formulations

For clinical use, the metabotropic glutamate receptor 5 antagonists are in accordance with the present invention suitably formulated into pharmaceutical formulations for oral administration. Also rectal, parenteral or any other route of administration may be contemplated to the skilled man in the art of formulations. Thus, the metabotropic glutamate receptor 5 antagonists are formulated with at least one pharmaceutically and pharmacologically acceptable carrier or adjuvant. The carrier may be in the form of a solid, semi-solid or liquid diluent.

In the preparation of oral pharmaceutical formulations in accordance with the invention, the metabotropic glutamate receptor 5 antagonist(s) to be formulated is mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or compressed into tablets.

Soft gelatine capsules may be prepared with capsules containing a mixture of the active compound or compounds of the invention, vegetable oil, fat, or other suitable vehicle for soft gelatine capsules. Hard gelatine capsules may contain the active compound in combination with solid powdered ingredients such as lactose, saccharose, sorbitol, mannitol, potato starch, corn starch, amylopectin, cellulose derivatives or gelatine.

Dosage units for rectal administration may be prepared (i) in the form of suppositories which contain the active substance(s) mixed with a neutral fat base; (ii) in the form of a gelatine rectal capsule which contains the active substance in a mixture with a vegetable oil, paraffin oil, or other suitable vehicle for gelatine rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the active compound and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of a compound of the invention in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

In one aspect of the present invention, the metabotropic glutamate receptor 5 antagonists may be administered once or twice daily, depending on the severity of the patient's condition.

Biological Evaluation

Methods

An in vivo gastric distension model is used as a model for functional gastrointestinal disorders, in particular for functional dyspepsia (Bayati A, Astin M, Ekman C, Mattsson H. Wistar Kyoto rats have impaired gastric adaptive accommodation in response to gastric distension. Gastroenterology 2003; 124 (4, suppl 1): W1471 (abstract)).

The gastric distension model enables detailed analysis of the physico-mechanical properties of the stomach, e.g. basal gastric tone, threshold for accommodation, accommodation rate, accommodation volume, and maximal gastric volume. By using the same model in both rats and humans it has been found that the gastric volume responses is very similar in the rat glandular stomach to that in human proximal stomach. Furthermore, it has been shown that patients with Functional Dyspepsia as well as Wistar Kyoto (WKY) rats have an impaired gastric adaptive response and also a lower total gastric volume as compared to healthy subjects and Sprague Dawley (SD) rats, respectively. In addition, the method has shown to be reproducible and reliable. Moreover, the advantage of the presently used barostat technique compared to other barostat techniques normally used in experimental clinical studies is that it is possible to discriminate between if a compound exerts its effect directly on gastric smooth muscles or if the effect involves the vagal reflex mechanism.

The rats are equipped with fistulas chronically implanted into the stomach. During gastric barostat experiments, a small inflatable plastic bag with a spherical shape is inserted through fistula into the glandular part of the stomach (middle to distal part in the rat). The experiments are performed in conscious rats. For detailed analysis of the physico-mechanical properties of the stomach, a combination of ramp and tonic distension paradigm is used. Pressure and volume data collected during experiments are saved for and further analysis.

In order to determine an animal's maximum gastric accommodation capacity, a balloon is inserted into the stomach of the animal and a four phase protocol which includes a start phase, a ramp phase, a tonic phase and an end phase is performed. A barostat system maintains the pressure by pumping air into and out of the balloon. The pressure applied to the balloon and the corresponding changes to the volume of the balloon are monitored throughout, e.g., using any barostat system known in the art (e.g., see Toma et al, Neurogastroenterol. Mot., 8, 19-28, 1996).

During the start phase a minimum distension pressure, e.g., 1 mmHg, is applied to the balloon until base line values are obtained. This is followed by a Ramp Phase. During this phase the pressure applied to the balloon is increased linearly with a constant increase in pressure to a preset maximum value. This second phase is then followed by the Tonic Phase at which the preset maximum pressure is kept constant. Finally, the pressure is rapidly dropped to the starting minimum distension pressure and this period is known as the End Phase.

To determine if an agent, e.g., a compound is useful in the treatment of FD, the maximum gastric accommodation rate in the WKY rat following administration of the compound is calculated. A compound of interest will be a compound that alters the maximum gastric accommodation rate in the animal and this is calculated by determining a difference in the maximum gastric accommodation rate before and after administration of the compound.

The Wistar Kyoto rats (WKY; M&B Denmark) are starved about 6 or 18 hours before each experiment depending on if the experiments are performed in the morning or in the afternoon. The balloon is inserted through the fistula into the stomach under isoflurane anaesthesia (Forene®, Abbott Scandinavia AB) and fixed in its position through the tightening of the fistula. The balloon that has a spherical shape with a wall thickness of about 15 μm, a non-distensible max diameter of 25 mm and a max volume of about 7 ml is connected to a polyethylene catheter with an outer diameter of 1.40 mm and a length of about 20 cm. After insertion of the balloon the animals are placed in specially designed Bollmann cages. The catheter from the balloon is then connected to a barostat system via a pressure transducer.

After the experiment the balloon and the connecting cable are removed under isoflurane anaesthesia and the animals are returned to their normal cages.

The ramp and tonic distension used in the WKY rat starts with a minimum distension pressure of 1 mmHg that continues for 20 min in order to collect base line values. The pressure is then increased by a velocity of 1 mmHg/min for 10 min to a maximum pressure of 10 mmHg (ramp phase). The barostat then keeps the pressure at the maximum pressure for 10 more min (tonic phase). After the tonic phase the pressure drops to the minimum distension pressure of 1 mmHg in about 1 s. The pressure is then kept at this level for another 20-minute period.

Results

MPEP

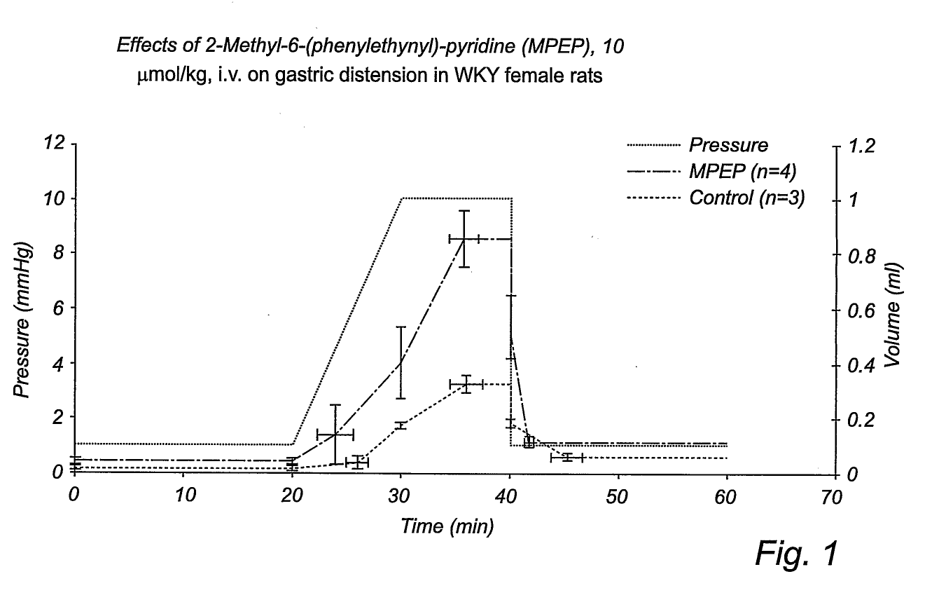

The metabotropic glutamate receptor subtype 5 (mGlu5) antagonist 2-Methyl-6-(phenylethynyl)-pyridine (MPEP) was administered intravenously into a tail vein approximately 3-5 min before start of the ramp phase. The experiment was carried out as described above. The result obtained is shown in FIG. 1.

The results shown in FIG. 1 show that MPEP in a dose of 10 μmol/kg in WKY rats induced an increase in the gastric volume both during the tonic phase and the ramp phase in addition to an increased maximum gastric volume compared to the control situation. The increased maximum gastric volume is probably due to the increased accommodation rate seen (the slope of volume curve during the tonic phase). The conclusion is thus that MPEP increases the accommodation capacity in WKY rats.

1-4. (canceled)

5. A method for the treatment of a functional gastrointestinal disorder comprising the administration of a therapeutically effective amount of a metabotropic glutamate receptor 5 antagonist, or a pharmaceutically acceptable salt or an optical isomer thereof, to a subject in need of such treatment.

6. The method according to claim 5, wherein the functional gastrointestinal disorder is functional dyspepsia.

7. The method according to any one of claim 5 or 6, wherein the metabotropic glutamate receptor 5 antagonist is 2-methyl-6-(phenylethynyl)-pyridine.

8. The method according to claim 7, wherein the metabotropic glutamate receptor 5 antagonist is a hydrochloride salt of 2-methyl-6-(phenylethynyl)-pyridine.
